(19) United States
(12) Patent Application Publication
(10) Pub. No.: US 2021/0052365 A1
(43) Pub. Date: Feb. 25, 2021

(54) IN SITU IMPLANTED BIONIC ARTIFICIAL ANAL SPHINCTER SYSTEM HAVING INDEPENDENT SWING ARM STRUCTURE

(71) Applicant: SHANGHAI JIAOTONG UNIVERSITY, Shanghai (CN)

(72) Inventors: Ding HAN, Shanghai (CN); Guozheng YAN, Shanghai (CN); Kai ZHAO, Shanghai (CN); Zhiwu WANG, Shanghai (CN); Zerun ZHOU, Shanghai (CN); Changjian WU, Shanghai (CN); Dasheng LIU, Shanghai (CN)

(21) Appl. No.: 16/963,819

(22) PCT Filed: Dec. 22, 2017
(86) PCT No.: PCT/CN2017/117895

§ 371 Date: Jul. 21, 2020

(30) Foreign Application Priority Data
Dec. 5, 2017 (CN) 201711264462.4

(51) Int. Cl. A61F 2/08; A61B 5/00; A61B 5/01

(52) U.S. Cl. CPC A61F 2/08; H02J 50/00; A61B 5/01; A61B 5/4255

(57) ABSTRACT

An in-situ implanted bionic artificial anal sphincter system with an independent swing arm structure comprises: an energy transmission control unit and a sphincter prosthesis unit. The energy transmission control unit comprises a master control module and a percutaneous wireless energy transmission module while the sphincter prosthesis unit comprises a wireless energy receiving module, a micro power supply, a sensor, an execution unit and a slave control module. The invention has a simpler and compact structure which reduces the risk of transplanting; the middle ring with independent rotating shaft has simple structure and reduces the difficulty of installation; the hall switch is arranged in the upper ring bin to facilitate wiring; the external gear meshing transmission torque is simple and reliable.

### TECHNICAL FIELD OF THE INVENTION

The present invention relates to a technology in the field of medical devices, in particular to an in-situ implanted bionic artificial anal sphincter system with an independent swing arm structure.

### TECHNOLOGY BACKGROUND

Artificial anal sphincter prosthesis simulates the physiological function of normal anal sphincter by sensing rectal pressure through pressure sensor, and remodels rectal sensory in combination with control system to realize autonomous control of defecation, thus treating fecal incontinence. The complexity of the existing fully closed anal sphincter prosthesis and the large number of parts lead to the large weight of the sphincter, which will lead to biocompatibility and safety problems after implantation; the difficulty in adjusting the installation position of Hall switch commonly used in the system makes it difficult to guarantee the normal working range of middle ring, which affects the normal opening or closing of the intestinal tract by the sphincter system; in addition, these prosthetic mechanisms generally have more middle rings and accessory components, and the assembly difficulty leads to the reduction of assembly accuracy, which affects the transmission effect, and then results in the energy consumption imbalance of sphincter system, affecting the normal operation of the system; the movable seal of the middle ring is difficult to guarantee, which may cause safety problems.

### BRIEF SUMMARY OF THE INVENTION

In view of the above shortcomings existing in the existing technology, an in-situ implanted bionic artificial anal sphincter system with an independent swing arm structure is proposed in the present invention. A planetary gear reducer is used in the present invention, which has a simpler and smaller structure and reduces the risk of surgical transplantation; the simple middle ring structure with independent rotating shaft reduces the installation difficulty; Hall switch is arranged within the upper ring compartment to facilitate wiring; the external gear meshing transmission torque is simple and reliable.

The present invention is realized by the following technical scheme:

The present invention comprises: an energy transmission control unit and a sphincter prosthesis unit. The energy transmission control unit comprises a master control module and a percutaneous wireless energy transmission module while the sphincter prosthesis unit comprises a wireless energy receiving module, a micro power supply, a sensor, an execution unit and a slave control module, wherein: master control module that is connected with the slave control module through wireless communication transmits the control command signal to the internal slave control module. The percutaneous wireless energy transmission module is connected with the wireless energy receiving module through electromagnetic coupling, of which the internal receiving module that is connected with the micro power supply transfers the external energy to the micro power supply for charging. The sensor and the actuator that are connected with the slave control module transmits the information on intestinal pressure, temperature and the position of the actuator, and the internal information is transmitted by the slave control module to the external control module.

The execution unit comprises the upper and lower ring mechanisms which are fixedly connected and opposite arranged, and the middle ring mechanism which is rotationally arranged between the upper ring mechanism and the lower ring mechanism, wherein: the fixed connection is realized by the driving mechanism which is connected with the upper ring mechanism and the lower ring mechanism respectively at each ends, and the middle ring mechanism can rotate through an independent rotating shaft connected between the upper and lower ring mechanisms. At least one end of the rotating shaft is meshed with the driving mechanism, and the output torque of the driving mechanism drives the swing arm movement of the middle ring mechanism through the rotating shaft.

The driving mechanism comprises a motor with a reduction box arranged inside the motor sleeve, wherein the output shaft of the reduction box is connected with at least one end of the rotating shaft through gear engagement.

The middle ring mechanism is fixedly connected with the rotating shaft, and a magnet is arranged in the magnet support which rotates synchronously with the rotating shaft, and a Hall element is arranged on the gear diaphragm in the corresponding upper ring mechanism bin. When the magnet rotates to the set position with the hall element, a limit signal is triggered to control the driving mechanism to stop working; when the driving mechanism reverses to drive the middle ring mechanism to rotate in the opposite direction, the position sensing of the hall element is also used to realize the limit. Furthermore, the exact position of the middle ring mechanism can be further determined by providing two hall elements.

### TECHNICAL EFFECTS

Compared with the existing technology, the technical effects of the invention include:

1) Simple mechanism, less number of parts, reduced the weight of the mechanism, strong compatibility and safety after implantation;

2) The hall element is arranged in the upper ring mechanism and is sealed by the sealing ring and the installation position of the Hall switch is easy to be adjusted to ensure the normal operation of the Hall switch, thus ensuring the normal opening or closing of the intestinal tract by the sphincter system.

3) The magnet matched with the hall element is arranged in the magnet support that rotates synchronously with the rotation axis, and the position can be adjusted according to the opening angle required by the swinging middle ring;

4) The overall size of the mechanism is reduced by using planetary gear reducer, and the motor sleeve can both fix the motor gear reducer and facilitate wiring.

5) Greatly shortened assembly time due to the fixed connection of middle ring mechanism with the rotating shaft and modular installation of the associated part the actuator, and the final integration into the upper and lower ring mechanism.

6) Movable sealing is guaranteed by adopting sealing ring for the middle ring mechanism.

### BRIEF DESCRIPTION OF THE SEVERAL VIEWS OF THE DRAWINGS

FIG. 1 is a schematic diagram of the artificial sphincter system of the present invention.

FIG. 2a is a schematic diagram of the overall structure explosion of the sphincter prosthesis;

FIG. 2b is a schematic diagram of the overall structure of the sphincter prosthesis.

FIG. 3 is the installation diagram of the rotating shaft of the invention;

FIG. 4 is the installation structure diagram of Hall element of the invention;

FIG. 5a is a schematic diagram of installation structure of the motor, reduction box and sleeve;

FIG. 6a is the side view of the installation structure of the motor, reduction box and sleeve;

FIG. 6a is the installation structure diagram of the independent middle ring mechanism of the invention;

FIG. 6b is a side view of the installation structure of the independent middle ring mechanism of the invention;

Where: 1 upper ring cover plate, 2 upper fixed ring, 3 gear cover plate, 4 gear diaphragm, 5 transmission gear train, 6 magnet support, 7 upper bearing sleeve cover plate, 8 upper bearing sleeve, 9 upper ring support, 10 reducer, 11 motor sleeve, 12 motor, 13 rotating shaft, 14 middle ring mechanism, 15 middle ring cover plate, 16 lower ring support, 17 lower bearing sleeve, 18 lower bearing sleeve cover plate, 19 lower fixed ring, 20 control communication module, 21 lower ring cover plate.

### DETAILED DESCRIPTION OF THE INVENTION

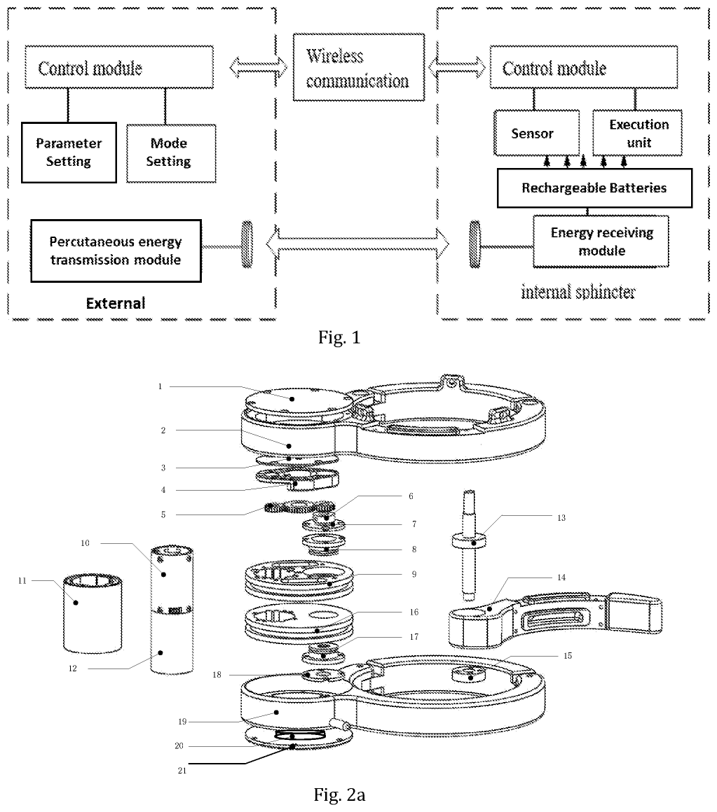

As shown in FIG. 1, the embodiment comprises an energy transmission control unit and a sphincter prosthesis unit. The energy transmission control unit comprises a master control module and a percutaneous wireless energy transmission module while the sphincter prosthesis unit comprises a wireless energy receiving module, a micro power supply, a sensor, an execution unit and a slave control module, wherein: master control module that is connected with the slave control module through wireless communication transmits the control command signal to the internal slave control module. The percutaneous wireless energy transmission module is connected with the wireless energy receiving module through electromagnetic coupling, of which the internal receiving module that is connected with the micro power supply transfers the external energy to the micro power supply for charging. The sensor and the actuator that are connected with the slave control module transmits the information on intestinal pressure, temperature and the position of the actuator, and the internal information is transmitted by the slave control module to the external control module.

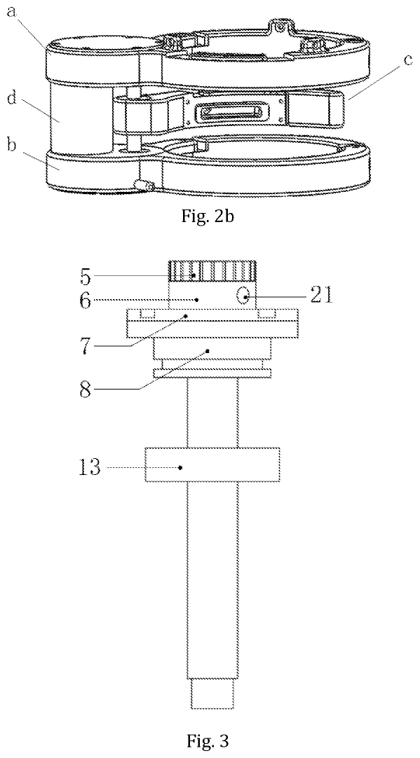

As shown in FIG. 2, the Execution unit comprises an upper ring mechanism a, a lower ring mechanism b and a middle ring mechanism c, wherein: the upper ring mechanism a is connected with the upper end of the driving mechanism d through the upper ring support 9, the lower ring mechanism b is connected with the lower end of the driving mechanism d through the lower ring support 16, the movable middle ring mechanism c is fixedly connected to an independent rotating shaft 13, and the upper end of the motor 12 in the motor sleeve 11 of the driving mechanism d is provided with a planetary gear reducer 10 through the two-stage transmission gear train 5 to transmit the torque to the independent rotating shaft 13, realizing the swing arm movement of the middle ring. And the swing angle is limited by the hall switch.

As shown in FIG. 3, the rotating shaft 13 is successively provided with a transmission gear train 5, a magnet support 6 and an upper bearing cover plate 7, which rotate synchronously with the rotating shaft 13. Wherein, a magnet is provided at the position hole 22 of the magnetic support 6, and the transmission gear train 5 is connected with the output shaft 10 of the reduction box of the driving mechanism d.

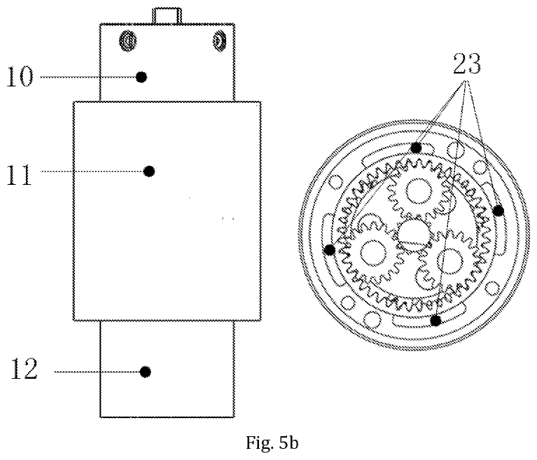

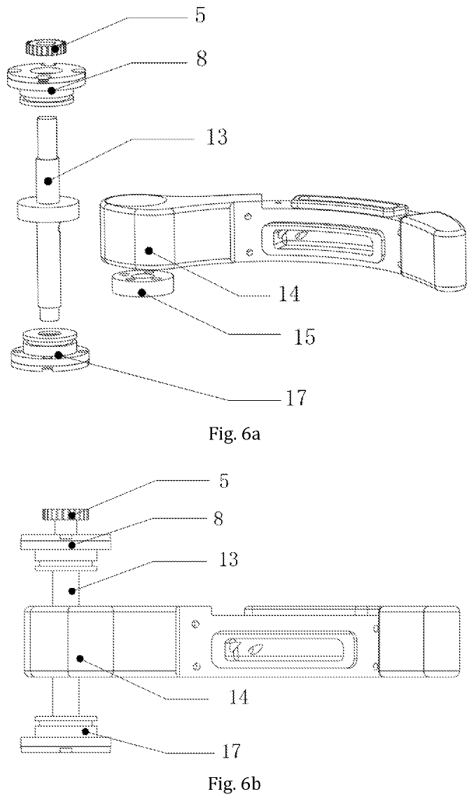

As shown in FIG. 5, both ends of the rotating shaft 13 are respectively provided with upper bearing sleeve 8 and lower bearing sleeve 17 to realize the rotational connection with the upper ring mechanism a and the lower ring mechanism b; the upper end of the rotating shaft 13 is of a D-shaped structure, so as to synchronously rotate with the transmission gear train 5 and transmit the torque to the middle ring mechanism c.

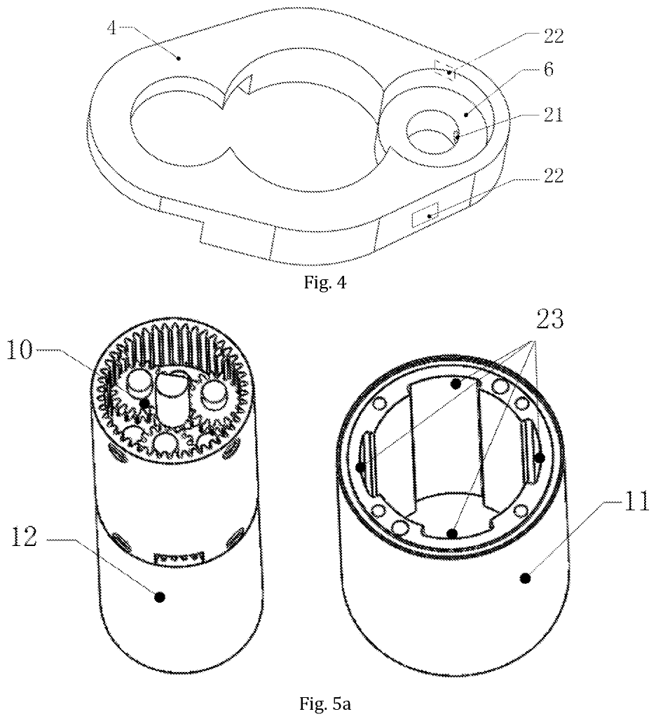

As shown in FIG. 4, the bottom of the upper ring mechanism a is provided with gear diaphragm 4, and the magnetic support 6 of the rotating shaft 13 is dynamically nested in gear diaphragm 4.

One side of the gear diaphragm 4 is provided with the position bin 23 for installing the Hall element. After the installation of the hall element, the hall element switch is formed by cooperating with the magnet at the position hole 22 to limit the middle ring mechanism: when the middle ring mechanism rotates, the magnets in the magnet position hole 6 will rotate together; when the magnet rotates to the position overlapped with the hall element, the hall element will generate a limit signal to prevent the driving mechanism from continuing to rotate, thus realizing the limit of the middle ring mechanism limit. Wherein, the design position of the magnet mounting position hole 6 and Hall element bin 23 is more open, which is convenient for adjusting and positioning.

As shown in FIG. 5, the reduction box 10 of the driving mechanism d is arranged above the motor 12 and nested in the motor sleeve 11. The motor sleeve 11 is provided with an arc groove 24, which connects wiring channel of lower ring control communication module 20 in correspondence to the wiring channel of the upper ring sensor and hall element.

The Mode of Carrying out the Invention above may be partially adjusted in different ways by a person skilled in the art on the premise of not deviating from the principle and purpose of the invention. The protection scope of the invention is subject to the claim and is not limited by the Mode of Carrying out the Invention above. Each Carrying out scheme within the scope of the invention is subject to the restriction of the invention.

What is claimed is:

1. An in-situ implanted bionic artificial anal sphincter system with an independent swing arm structure is characterized by comprising an energy transmission control unit and a sphincter prosthesis unit; the energy transmission control unit comprises a master control module and a percutaneous wireless energy transmission module while the sphincter prosthesis unit comprises a wireless energy receiving module, a micro power supply, a sensor, an execution unit and a slave control module, wherein: master control module that is connected with the slave control module through wireless communication transmits the control command signal to the internal slave control module, the percutaneous wireless energy transmission module is connected with the wireless energy receiving module through electromagnetic coupling, of which the internal receiving module that is connected with the micro power supply transfers the external energy to the micro power supply and charges it; the sensor and the actuator that are connected with the slave control module transmits the information on intestinal pressure, temperature and the position of the actuator, and the internal information is transmitted by the slave control module to the external control module;
the execution unit comprises: the upper and lower ring mechanisms which are fixedly connected and opposite arranged, and the middle ring mechanism which is rotationally arranged between the upper ring mechanism and the lower ring mechanism, wherein: the fixed connection is realized by the driving mechanism which is connected with the upper ring mechanism and the lower ring mechanism respectively at both ends, and the middle ring mechanism can rotate through an independent rotating shaft connected between the upper and lower ring mechanisms; at least one end of the rotating shaft is meshed with the driving mechanism, and the output torque of the driving mechanism drives the swing arm movement of the middle ring mechanism through the rotating shaft;
the middle ring mechanism is fixedly connected with the rotating shaft, and a magnet is arranged in the magnet support which rotates synchronously with the rotating shaft, and a hall element is arranged on the gear diaphragm in the corresponding upper ring mechanism bin.; when the magnet rotates to the set position with the hall element, a limit signal is triggered to control the driving mechanism to stop working; when the driving mechanism reverses to drive the middle ring mechanism to rotate in the opposite direction, the position sensing of the hall element is also used to realize the limit.

2. The artificial anal sphincter system as described in claim 1 is characterized by a control circuit board and a communication receiving module installed in the lower ring mechanism.

3. The artificial anal sphincter system as described in claim 1 is characterized by the adoption of O-ring sealing between the parts mechanism.

4. The artificial anal sphincter system as described in claim 1 is characterized by sensors that are arranged on the upper, middle and lower rings respectively to sense radial and axial pressure in the gut.

5. The artificial anal sphincter system as described in claim 3 is characterized by the gel in the pressure sensor bin that is injected through the injection hole.

6. The artificial anal sphincter system as described in claim 1 is characterized by the driving mechanism that comprises a motor with a reduction box arranged inside the motor sleeve, wherein the output shaft of the reduction box is connected with at least one end of the rotating shaft through gear engagement.

7. The artificial anal sphincter system as described in claim 1 is characterized by the rotating shaft that are provided with driving gear train, the magnet support and the upper bearing cover plate are arranged on the rotating shaft in turn, which rotate synchronously with the rotating shaft. Wherein, the magnet support is provided with a position hole for installing the magnet, and the driving gear train is connected with the output shaft of the reduction box of the driving mechanism.

8. The artificial anal sphincter system as described in claim 3 is characterized by the upper bearing sleeve and lower bearing sleeve that are arranged on each end of the rotating shaft to realize the rotary connection with the upper ring mechanism and the lower ring mechanism; The upper end of the rotating shaft is a D-shaped structure, which rotates synchronously with the transmission gear train and transmits the torque to the middle ring mechanism.

9. The artificial anal sphincter system as described in claim 3 is characterized by the gear diaphragm that is arranged on the bottom of the upper ring mechanism, and the magnet support that is movably nested in the gear diaphragm.

10. The artificial anal sphincter system as described in claim 3 is characterized in that one side of the gear diaphragm is provided with a position bin for installing the hall element; the hall element forms a hall element switch by cooperating with the magnet at the position hole to limit the middle ring mechanism: when the middle ring mechanism rotates and drives the magnet to move synchronously to the position that coincides with the hall element, the hall element is triggered to generate the limit signal that will prevent the driving mechanism from continuing to rotate, so as to realize the limit of the middle ring mechanism.

11. The artificial anal sphincter system as described in claim 1 is characterized in that the reducer of the driving mechanism is arranged above the motor and nested in the motor sleeve, the motor sleeve is provided with an arc groove, and the channel of corresponding position hole is arranged in the rotation shaft, thus forming the wiring channel of hall element.

12. The artificial anal sphincter system as described in claim 1 is characterized by the least two hall elements that are provided to further determine the exact location of the middle ring mechanism.
